(19) United States
(12) Patent Application Publication
(10) Pub. No.: US 2010/0254936 A1
(43) Pub. Date: Oct. 7, 2010

(54) COMPOSTIONS AND METHODS TO CONTROL FUNGAL PATHOGENS

(75) Inventors: Norman R. Pearson, Carmel, IN (US); Ronald Ross, JR., Zionsville, IN (US); Robert J. Ehr, Indianapolis, IN (US)

Correspondence Address: DOW AGROSCIENCES LLC, 9330 ZIONSVILLE RD, INDIANAPOLIS, IN, 46268, US

(73) Assignee: Dow AgroSciences LLC, Indianapolis, IN (US)

(21) Appl. No.: 12/416,764

(22) Filed: Apr. 1, 2009

(51) Int. Cl. A01N 33/26; C07C 243/28; A01P 19/00

(52) U.S. Cl. 424/84; 564/151; 564/149

(57) ABSTRACT

Disclosed herein are acylhydrazone and semicarbazones derivatives of aldehydes and ketones that may act to attract plant pathogenic zoospores and methods of using these compounds. These compounds include the compound according to Formula 1:

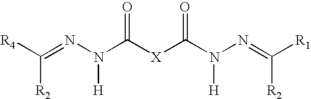

wherein: X is selected from the group consisting of: (CH₂)ₙ, 1,3-phenylene and 1,4-phenylene; R₁ is selected from the group consisting of iso-butyl, sec-butyl and tert-butyl-CH₂; R₂ is hydrogen or methyl; and n is equal to 0-25. Upon exposure to water, these compounds release aldehydes or ketones that may attract zoospores. These compounds can be combined with fungicides to form fungicidal formulations that are especially effective against oomycete producing fungal pathogens.

### FIELD OF THE INVENTION

Various aspects disclosed herein relate to new fungal zoospore attractant releasing compositions which may be well suited for formulation with fungicides in various compositions that may be used in methods of controlling fungi.

### BACKGROUND

Economically important plants may be attacked by a diverse range of plant pathogens. Many of the resulting diseases are caused by oomycete pseudo fungi. Many of the diseases caused by oomycete pathogens such as late blight of potato or tomato, grape downy mildew or downy mildew diseases of vegetables can be especially damaging.

The life cycle of many oomycete pathogens involves a series of spore forms that are very important in the spread and propagation of these diseases. For example, during the asexual life cycle of a number of oomycete pseudo-fungi, such as Phytophthora infestans, the cause of late blight of potatoes, and Plasmopara viticola, which causes downy mildew of grapes, non-motile spores called sporangia are produced by the fungal pathogen. Under suitable conditions, the contents of sporangia form additional spores called zoospores. Zoospores have flagella and are capable of swimming in water, i.e. they are motile. Zoospores serve as major infection agents by swimming to and encysting near the stomata of a plant or other suitable places on the leaf, stem, root, seed or tuber for infecting the plant. On foliage, the stomata are then entered into by germ tubes from the germinating cysts or in some cases the germ tube from the encysted zoospore can directly penetrate the leaf or root surface.

Researchers have identified various chemicals known to attract fungal zoospores. These zoospore attractants may generally be described as a substance or compound that causes a chemotactic response by a zoospore. Examples of some zoospore attractant chemicals are disclosed in the article “Fatty acids, aldehydes and alcohols as attractants for zoospores of Phytophthora palmivora” in Nature, volume 217, page 448, by Cameron and Carlile. Further examples of zoospore attractants may be found in the articles “Biology of Phytophthora zoospores” in Phytopathology, volume 60, pages 1128-1135 by Hickman and “Chemotactic response of zoospores of five species of Phytophthora” in Phytopathology, volume 63, page 1511 by Khew. The disclosures of each of the above mentioned articles are expressly incorporated by reference herein.

Generally, these zoospore attractant chemicals or substances are produced by the root region of plants and may enhance the infection process in the rhizosphere by enabling the zoospores to locate a point for infection. It is possible that plant foliage or specific sites on the foliage also produce substances that are attractive to zoospores. Substances have been tested for their ability to attract zoospores through chemotaxis using a variety of published methods such as those employing capillary tubes that emanate the substance to be tested. Such methods are generally applicable and are described in various publications, including:
1. Donaldson, S. P. and J. W. Deacon. 1993. New Phytologist, 123: 289-295.
2. Tyler, B. M., M-H. Wu, J-M. Wang, W. Cheung and P. F. Morris. 1996. Applied and Environmental Microbiology, 62: 2811-2817.
3. Khew, K. I. and G. A. Zentmeyer. 1973. Phytopathology, 63: 1511-1517.

Generally, compounds to be tested for their ability to attract zoospores through chemotaxis must have sufficient water solubility or, if of low water solubility, they must be in a suitable physical form or matrix to allow sufficient release of the test compound. Researchers have found that certain naturally occurring or synthetic short chain aldehydes and ketones (C4-C8) are potent zoospore attractants. It has been further demonstrated that these aldehydes and ketones enhance the effectiveness of fungicides having inherent effects on oomycete pathogens when applied in mixture with them. However, the utility of most short chain aldehydes and ketones for this purpose is limited by certain physical properties such as high volatility or high water solubility.

The present disclosure provides new methods and compositions of controlling oomycete fungal plant pathogens. The inventive composition typically comprises a composition suitable for controlling oomycete fungi capable of producing zoospores, the composition including an agriculturally effective amount of a fungicide and at least one zoospore attractant derivative derived from a short chain aldehyde or ketone zoospore attractant molecule.

### SUMMARY

One embodiment is a composition suitable for attracting zoospores of oomycete fungi. The derivative hydrolyzes in water over time to release a zoospore attractant. A further embodiment is a mixture of these zoospore attractant derivatives and an agriculturally effective amount of a fungicide effective on oomycete fungi.

One embodiment is a zoospore attractant derivative that upon exposure to water releases a zoospore attractant. These attractant derivatives are generally either acylhydrazones or semicarbazones and include compounds selected from the group consisting of Formula 1, Formula 2, Formula 3, Formula 4, Formula 5, and Formula 6 wherein, Formula 1 is

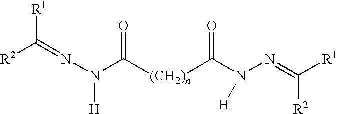

wherein, R₁=iso-butyl, sec-butyl, or tert-butyl-CH₂; R₂═H; and n=0-25; Formula 2 is:

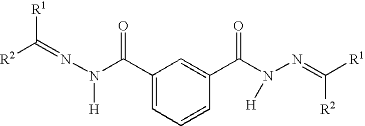

wherein, R₁=sec-butyl, or tert-butyl-CH₂; and R₂═H; Formula 3 is:

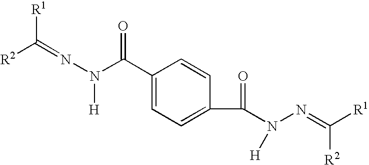

wherein, R₁=iso-butyl, sec-butyl, or tert-butyl-CH₂; and R₂═H; Formula 4 is:

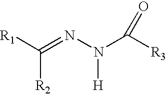

wherein, R₁=iso-butyl, R₂═H; and R₃=an n-alkyl including 2 -25 carbons, excluding n-heptyl or n-undecyl alkyls, or a branched-alkyl including 4-25 carbons, or a substituted or unsubstituted cycloalkyl including 3-25 carbons, or a substituted or unsubstituted arylalkyl including 12-26 carbons; or R₁=sec-butyl, or tert-butyl-CH₂; R₂═H; and R₃=an n-alkyl including 1-25 carbons, or a branched-alkyl including 3-25 carbons, or a substituted or unsubstituted cycloalkyl including 7-25 carbons, or a substituted or unsubstituted arylalkyl including 7-25 carbons, or R₁=iso-butyl, R₂=methyl; and R₃=an n-alkyl including 2, 3, 6 and 12-25 carbons, or a branched-alkyl including 3-25 carbons, or a substituted or unsubstituted cycloalkyl including 3-25 carbons, or a substituted or unsubstituted arylalkyl including 7-25 carbons; Formula 5 is

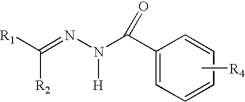

wherein, R₄═H, alkyl or haloalkyl or alkoxy or alkylthio, or haloalkoxy or haloalkylthio, each including 1-4 carbons, or halo, hydroxyl, nitro, carboxyl acid, carboxylic acid derivatives or cyano; and either R₁=sec-butyl, or tert-butyl-CH₂; and R₂═H; or R₁=iso-butyl; and R₂=methyl; or Formula 6 is:

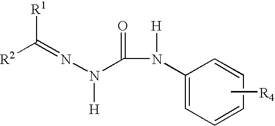

wherein; R₄=H, alkyl or haloalkyl or alkoxy or alkylthio, or haloalkoxy or haloalkylthio, each including 1-4 carbons, or halo, hydroxyl, nitro, carboxyl acid, carboxylic acid derivatives or cyano; and either R₁=iso-butyl, sec-butyl, or tert-butyl-CH₂; and R₂═H; or R₁=iso-butyl; and R₂=methyl.

Another embodiment is a method for controlling a fungal pathogen, comprising the steps of: applying at least one composition according to Formulae 1-6, mixed with an agriculturally effective amount of a fungicide to an area adjacent to the fungal pathogen. In one embodiment the fungal pathogen is an oomycete fungal pathogen and the above mixture is applied to plant tissue before the fungal pathogen has initiated an infection.

Yet another embodiment is a formulation for the control of a fungus, comprising: at least one compound selected from the group including Formulas 1-6 and at least one fungicide. In one embodiment the fungicide is effective against a fungus that produces motile zoospores. In one embodiment one or more fungicides is selected from the group consisting of: mancozeb, maneb, zineb, thiram, propineb, metiram, copper hydroxide, copper oxychloride, Bordeaux mixture, captan, folpet, amisulbrom, azoxystrobin, trifloxystrobin, picoxystrobin, kresoxim-methyl, fluoxastrobin, pyraclostrobin, famoxadone, fenamidone, metalaxyl, mefenoxam, benalaxyl, cymoxanil, propamocarb, dimethomorph, flumorph, mandipropamid, iprovalicarb, benthiavalicarb-isopropyl, valiphenal, zoxamide, ethaboxam, cyazofamid, fluopicolide, fluazinam, chlorothalonil, dithianon, fosetyl-Al, phosphorous acid, tolylfluanid, and 4-fluorophenyl(1S)-1-({[(1R,S)-(4-cyanophenyl)ethyl]sulfonyl}methyl)propylcarbamate. In one embodiment the fungicide is a non-copper based fungicide. In a further embodiment the fungicide may be a compound of Formula I wherein:

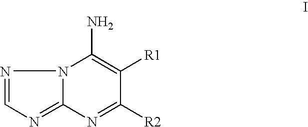

wherein R1 is ethyl, 1-octyl, 1-nonyl, or 3,5,5-trimethyl-1-hexyl and R2 is methyl, ethyl, 1-propyl, 1-octyl, trifluoromethyl, or methoxymethyl.

Still another embodiment is a method for controlling a fungal infestation, comprising the steps of: providing at least one formulation including at least one compound selected from Formulae 1-6, at least one fungicide and applying an agriculturally effective amount of the formulation or mixture to an area adjacent to a fungus. In one embodiment the mixture including the attractant derivative and fungicide is applied to at least one of the plant, plant foliage, blossoms, stems, fruits, the area adjacent to the plant, soil, seeds, germinating seeds, roots, liquid and solid growth media, and hydroponic growth solutions.

In one embodiment the formulation, comprised of at least one zoospore attractant derivative and at least one fungicide effective on oomycete fungal pathogens, is adapted to control diseases caused by oomycete fungal pathogens selected from the group consisting of Phytophthora infestans, Plasmopara viticola, Phytophthora capsici, Pseudoperonospora cubensis, Bremia lactucae, Phytophthora phaseoli, Phytophthora nicotiane var. parasitica, Sclerospora graminicola, Sclerophthora rayssiae, Phytophthora palmivora, Phytophthora citrophora, Sclerophthora macrospora, Sclerophthora graminicola, Phytophthora cactorum, Phytophthora syringe, Pseudoperonospora humuli, and Albugo candida.

In another embodiment the zoospore attractant derivative may release zoospore attractants such as C4-C8 aldehydes or ketones selected from the group consisting of isovaleraldehyde, 2-methylbutyraldehyde, valeraldehyde, isobutyraldehyde, butyraldehyde, 4-methylpentanal, 3,3-dimethylbutyraldehyde, 3-methylthiobutyraldehyde, 2-cyclopropylacetaldehyde, 3-methylcrotonaldehyde, 2-ethylcrotonaldehyde, crotonaldehyde, 2-methylcrotonaldehyde, 3-indolecarbaldehyde, furfural(2-furaldehyde), 2-thiophenecarboxaldehyde, 2-ethylbutyraldehyde, cyclopropanecarboxaldehyde, 2,3-dimethylvaleraldehyde, 2-methylvaleraldehyde, tetrahydrofuran-3-carboxaldehyde, cyclopentanecarboxaldehyde, 3-methyl-2-pentanone, 4,4-dimethyl-2-pentanone, 3,3-dimethyl-2-butanone, and 4-methyl-2-pentanone.

It is envisioned that substances that induce encystment of zoospores, such as pectin, a metal ion, and an inorganic compound or inorganic salt compound selected from the group consisting of Ca, Zn, Mg, Mn, NaNO₃, KNO₃, and NaCl, may be added to compositions containing a fungicide and a zoospore attractant derivative to further improve disease control.

### BRIEF DESCRIPTION OF THE FIGURES

FIG. 1 shows some acylhydrazone and semicarbazone derivatives of aldehydes and ketones used in accordance with the methods disclosed herein and tested for their ability to attract zoospores.

FIG. 2 shows a summary of the data collected by testing the ability of various acylhydrazone and semicarbazone derivatives of aldehydes and ketones to attract motile zoo spores of Phytophthora capsici (PHYTCA) and Plasmopara viticola (PLASVI).

### DESCRIPTION

For the purposes of promoting an understanding of the principles of the novel technology, reference will now be made to the various exemplary embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates.

The present invention relates to compounds that are or may form derivatives of zoospore attractant compounds that release zoospore attractants and may be used to increase the efficacy of fungicides for controlling plant diseases caused by oomycete pathogens. The inventive methods comprise contacting a plant at risk of being diseased from an oomycete pathogen that produces zoospores with a composition comprising an effective amount of a fungicide and a zoospore attractant derivative. Alternatively, a mixture of differing zoospore attractants and zoospore attractant derivatives may be used with a fungicide or a mixture of differing fungicides.

While not wishing to be bound by any theory it is believed that embedding, coating or surrounding a fungicide particle with a zoospore attractant derivative to create a concentration gradient of a zoospore attractant around the fungicide particle that attracts zoospores toward the fungicide, could enhance the effectiveness of the composition. By attracting the zoospores to the fungicide particle, the area of disease control of the fungicide may be increased, possibly lowering the use rate of the fungicide or extending the period of disease control. Additionally, a broader range of fungicides may be used, including fungicides that have limited redistribution on the plant surface.

While not wishing to be bound by any theory it is believed that using a zoospore attractant derivative could enhance the effectiveness of zoospore active fungicides such as thiocarbamates, mancozeb, maneb, zineb, thiram, propineb, or metiram; copper-based fungicides such as copper hydroxide, copper oxychloride, or Bordeaux mixture; phthalimide fungicides such as captan or folpet; amisulbrom; strobilurins such as azoxystrobin, trifloxystrobin, picoxystrobin, kresoxim-methyl, fluoxastrobin, pyraclostrobin and others; famoxadone; fenamidone; metalaxyl; mefenoxam; benalaxyl; cymoxanil; propamocarb; dimethomorph; flumorph; mandipropamid; iprovalicarb; benthiavalicarb-isopropyl; valiphenal, zoxamide; ethaboxam; cyazofamid; fluopicolide; fluazinam; chlorothalonil; dithianon; fosetyl-AL, phosphorous acid; tolylfluanid, or aminosulfones such as 4-fluorophenyl(1S)-1-({[(1R,S)-(4-cyanophenyl)ethyl]sulfonyl}methyl)propylcarbamate or the following triazolopyrimidine compounds such as those shown by Formula I:

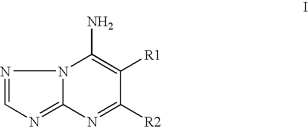

wherein R1 is ethyl, 1-octyl, 1-nonyl, or 3,5,5-trimethyl-1-hexyl and R2 is methyl, ethyl, 1-propyl, 1-octyl, trifluoromethyl, or methoxymethyl.

Useful zoospore attractant derivatives and the attractants they release may vary depending upon the type of plant, the fungal pathogen and environmental conditions. Typical zoospore attractants may include, for example, derivatives of C4-C8 aldehydes or C4-C8 ketones. Zoospore attractant derivatives may also be absorbed onto or embedded into an inert substrate such as PergoPak M, corn starch, clay, latex agglomerates, or fertilizer particles.

Zoospore attractant derivatives may be used for purposes such as controlled release of the attractant molecule. Zoospore attractant derivatives are chemical compounds generally made or derived from zoospore attractant molecules. Zoospore attractant derivatives may be used in combination with zoospore attractants and fungicides. Suitable zoospore attractant derivatives such as acylhydrazone derivatives or semicarbazone derivatives of various aldehydes and ketones may be less volatile and/or water soluble than their corresponding aldehyde or ketone. These derivatives may produce or release zoospore attractants once the derivative comes into contact with water on a plant surface or the area adjacent to the plant. Examples of hydrazone derivative technology are included in PCT Patent Application No. WO2006016248 and the article entitled “Controlled release of volatile aldehydes and ketones by reversible hydrazone formation—‘classical’ profragrances are getting dynamic” by Levrand et al. published in Chemical Communications (Cambridge, United Kingdom) (2006) on pages 2965-2967 (ISSN: 1359-7345). The disclosure of each of the above references is hereby expressly incorporated by reference herein. Various methods for the synthesis of novel and or useful acylhydrazone and semicarbazone derivatives are provided herein.

Reaction 1, shown below, describes how a generic zoospore attractant derivative, such as those shown above in Formulae 1-6, releases the zoospore attractant upon contact with water. The rate of production of the zoospore attractant is dependent on many factors, among them: the physico-chemical properties of the zoospore attractant derivative, the composition of the formulation containing the derivative, the presence and duration of water on the plant surface, and the temperature, humidity and other environmental conditions in the area of the application. As shown, this hydrolysis reaction is theoretically reversible, but this reversibility under practical agricultural conditions is likely to be low.

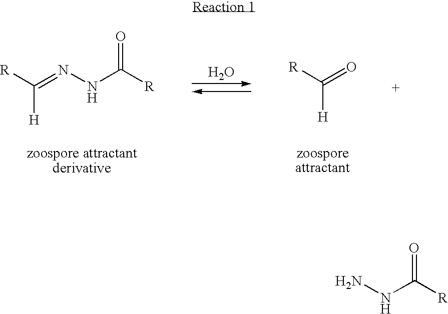

The aforementioned zoospore attractant derivatives when used in combination with fungicides may provide particularly effective control of diseases caused by the pathogens Phytophthora infestans, Plasmopara viticola, Phytophthora capsici, and Pseudoperonospora cubensis. Other pathogens that may also be controlled on a variety of plants such as tomatoes, potatoes, peppers, grapes, cucurbits, lettuce, beans, sorghum, corn, citrus, turf grasses, pecans, apples, pears, hops, and crucifiers include: Bremia lactucae, Phytophthora phaseoli, Phytophthora nicotiane var. parasitica, Sclerospora graminicola, Sclerophthora rayssiae, Phytophthora palmivora, Phytophthora citrophora, Sclerophthora macrospora, Sclerophthora graminicola, Phytophthora cactorum, Phytophthora syringe, Pseudoperonospora humuli, and Albugo candida.

The effective amount of the zoospore attractant derivative to be employed with the fungicide often depends upon, for example, the type of plants, the stage of growth of the plant, severity of environmental conditions, the fungal pathogen and application conditions. Typically, a plant in need of fungal protection, control or elimination is contacted with an amount of zoospore attractant derivative from about 1 to about 5000 ppm, preferably from about 10 to about 1000 ppm of the zoospore attractant derivative. The contacting may be in any effective manner. For example, any exposed part of the plant, e.g., leaves or stems may be sprayed with the attractant derivative in mixture with effective rates of a fungicide. The attractant derivative may be formulated by itself in an agriculturally suitable carrier and comprise 1 to 95% by weight of the formulation. One or more attractant derivatives may be co-formulated with one or more zoospore attractants and one or more fungicides as a liquid or a solid wherein the attractant, attractant derivative, or mixture of one or more attractants or attractant derivatives comprises 1 to 50% of the formulation.

The aforementioned zoospore attractant derivative enhanced fungicides may be applied to the plant foliage or the soil or area adjacent to the plant. Additionally, the zoospore attractant derivative enhanced fungicides may be mixed with or applied with any combination of herbicides, insecticides, bacteriocides, nematocides, miticides, biocides, termiticides, rodenticides, molluscides, arthropodicides, fertilizers, growth regulators, and pheromones.

### Experimental Section

The general scheme for the synthesis of the various acylhydrazone and semicarbazone derivatives of several of the aldehydes and ketones of the present disclosure is shown below in Scheme 1.

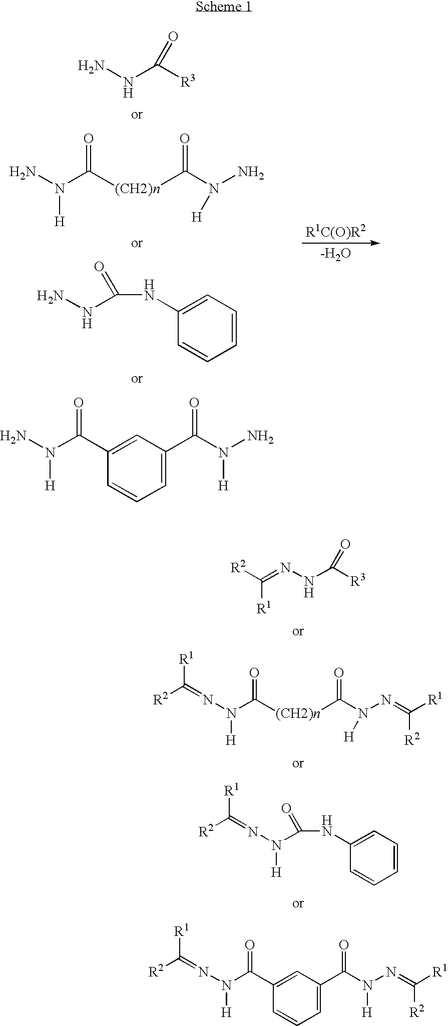

For Scheme 1, R¹ is an alkyl, R² is a hydrogen or a methyl, R³ is an aliphatic group and n is 0-8.

### Synthesis of Compounds 10 and 12

Briefly, a mixture of 10.0 g (51.5 mmol) of isophthalic acid bis-hydrazide, 150 mL of absolute ethanol and 124 millimoles of the aldehyde, was heated at reflux for 8-24 hours. Analysis by TLC indicated complete consumption of the starting hydrazide. The mixture was allowed to cool to room temperature over many hours and was then filtered. The white solid obtained was washed with ethanol and then dried to constant weight in a vacuum oven at 40-50° C. The isolated solid was analyzed by proton NMR spectroscopy and by elemental analysis and the results of these analyses were consistent with the assigned structure. The melting point was also determined.

### Synthesis of Compound 5

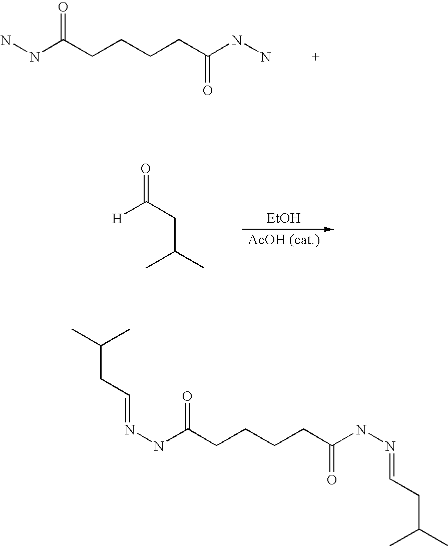

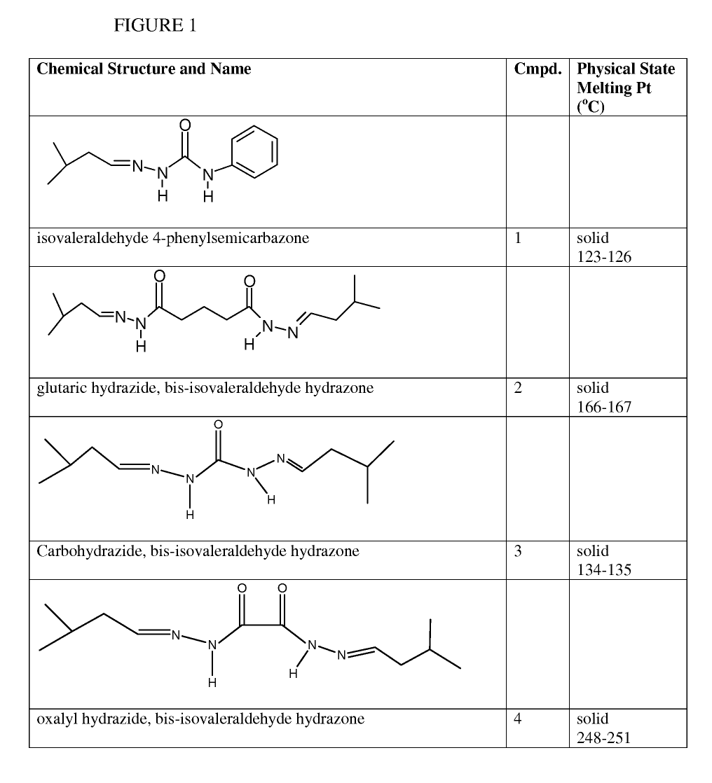

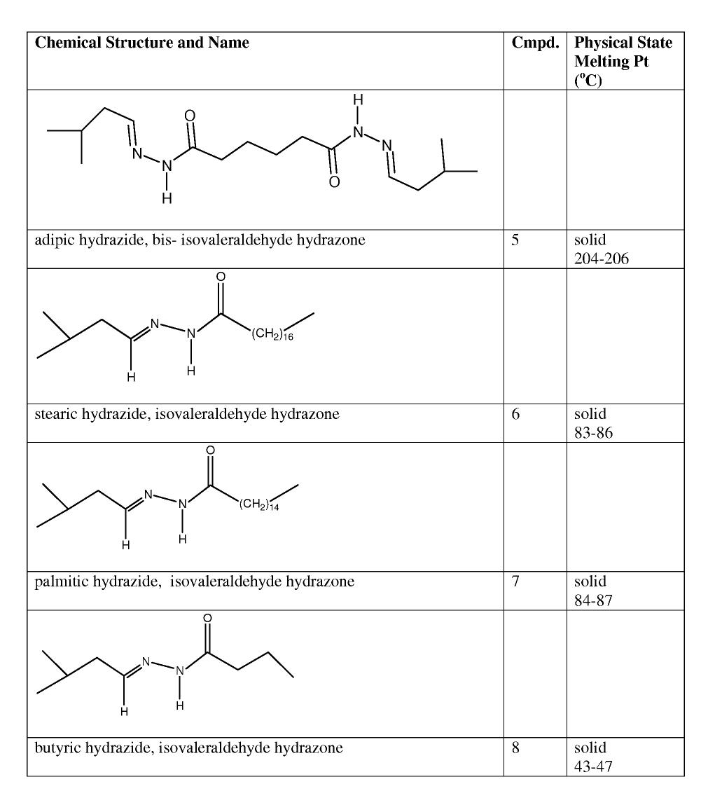

Briefly, a dry 250 ml round bottom flask equipped with magnetic stirrer, thermometer, and reflux condenser was charged the 10.0 g (57.4 mmol) of adipic acid dihydrazide, 0.5 mL of glacial acetic acid and 150 mL of absolute ethanol. After most of the solid dissolved, 13.5 mL (126 mmol) of isovaleraldehyde was added to the flask and it was then heated at reflux for 4 hours. The progress of the reaction was checked by drawing an aliquot of the reaction mixture and analyzing it by HPLC. Once the reaction had proceeded nearly to completion, the mixture was cooled to room temperature. The resulting solid was collected by vacuum filtration, rinsed with hexanes and dried in vacuuo at 40° C. About, 16 g of white solid product was isolated (90% yield). The structure of the product was consistent with the structure of compound 5 as determined by analysis by 300 MHz ¹H NMR and HPLC/MS. The melting point was determined to be 204-206° C. Similar methods were used to produce compounds 1 through 9 and compound 11 in FIG. 1.

Design of Zoospore Attractant Activity Assay

Referring now to FIG. 1, the compounds listed therein were evaluated for their ability to attract motile zoospores of two different fungi Phytophthora capsici (PHYTCA) and Plasmopara viticola (PLASVI). Compounds with sufficient water solubility were tested as 5 mM solutions. Compounds with insufficient water solubility were milled and formulated as 10% suspension concentrates. Solutions for suspension concentrates were placed in 1.0 cm Drummond Size 2 microcap capillary tubes. Tubes were placed in the well of a 12-well plate and held in place with a small drop of Dow Corning vacuum grease.

Briefly, for use in these assays, Phytophthora capsici was grown on V-8 agar. When the culture was 5-7 days old and producing abundant sporangia, production of zoospores was initiated by adding 15 ml of sterile water to the plate. After 10 minutes at room temperature, the flooded plate was placed in a refrigerator at 4° C. for 20 minutes. Then the plate was returned to room temperature for 30-60 minutes. The zoospore suspension was then filtered through Whatman 113V Filter paper.

In order to initiate production of Plasmopara viticola zoospores, sporangia were harvested from three leaves of grapes (Vitis vinifera cv Carignane) that were completely infected and producing abundant sporulation. The infected leaves were placed in de-ionized water and sporangia were dislodged by brushing the leaf lightly. The solution including the dislodged sporangia was allowed to sit at room temperature for 10 minutes. The solution was transferred to a refrigerator at 4° C. for 20 minutes and then returned to room temperature for 60 to 90 minutes, at which time large numbers of zoospores were present. The zoospore suspension then was filtered through Whatman 113V Filter paper.

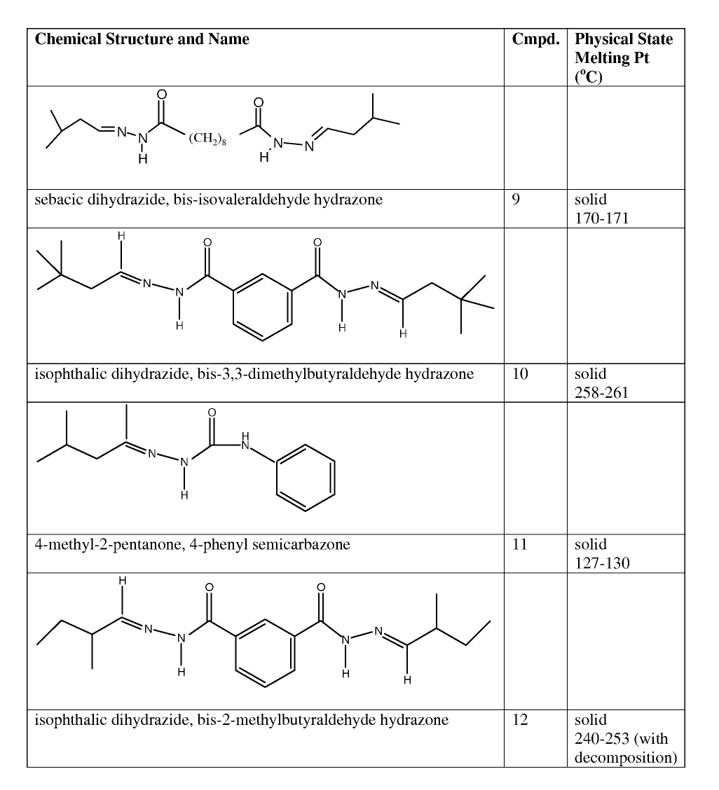

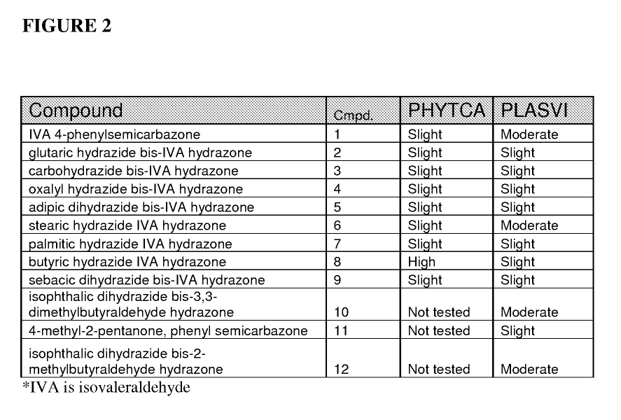

Approximately 1 ml of zoospore suspension was placed in the wells containing the capillary tubes of chemicals to be tested for attractant activity. After 60 to 90 minutes the level of attractancy was scored by viewing the capillary tube and solution under a microscope and comparing the number of zoospores that had swum into the capillary tube with the number in the external solution. That ratio was scored semi-quantitatively on a scale of 1 to 10, with 1 representing no accumulation of zoospores inside the capillary tube, with 2 representing an accumulation of zoospores inside the capillary tube about equal to the zoospore density in the external solution, with 5 representing an accumulation about 5 fold the density in the external solution and 10 representing a capillary full of zoospores at a density too numerous to count. Average scores of 1-3 were categorized as Slight Attractancy; average scores of 4-7 were categorized as Moderate Attractancy; average scores of 8-10 were categorized as High Attractancy. These results are summarized in FIG. 2. As an alternative to this method, the procedures for determining zoospore attraction referenced in the Background are generally applicable and may also be employed.

Compounds of the invention having zoospore attractant properties may be advantageously combined with a number of fungicides especially fungicides that are active against oomycete fungal pathogens. Useful fungicides include the group consisting of mancozeb, maneb, zineb, thiram, propineb, metiram, copper hydroxide, copper oxychloride, Bordeaux mixture, captan, folpet, amisulbrom, azoxystrobin, trifloxystrobin, picoxystrobin, kresoxim-methyl, fluoxastrobin, pyraclostrobin, famoxadone, fenamidone, metalaxyl, mefenoxam, benalaxyl, cymoxanil, propamocarb, dimethomorph, flumorph, mandipropamid, iprovalicarb, benthiavalicarb-isopropyl, valiphenal, zoxamide, ethaboxam, cyazofamid, fluopicolide, fluazinam, chlorothalonil, dithianon, fosetyl-AL, phosphorous acid, tolylfluanid, 4-fluorophenyl(1S)-1-({[(1R,S)-(4-cyanophenyl)ethyl]sulfonyl}methyl)propylcarbamate and triazolopyrimidine compounds such as those shown by Formula I:

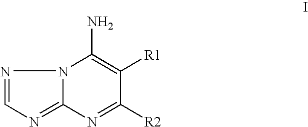

wherein R1 is ethyl, 1-octyl, 1-nonyl, or 3,5,5-trimethyl-1-hexyl and R2 is methyl, ethyl, 1-propyl, 1-octyl, trifluoromethyl, or methoxymethyl.

The compounds of this invention are preferably applied in the form of a composition comprising one or more of the compounds of Formulae 1-6 with a phytologically-acceptable carrier. The compositions are either concentrated formulations which are dispersed in water or another liquid for application, or are dust or granular formulations which are applied without further treatment. The compositions are prepared according to procedures which are conventional in the agricultural chemical art, but which are novel and important because of the presence therein of the compounds of this invention. Some description of the formulation of the compositions is given to assure that agricultural chemists can readily prepare desired compositions.

The dispersions in which the compounds are applied are most often aqueous suspensions or emulsions prepared from concentrated formulations of the compounds. Such water-soluble, water suspendable, or emulsifiable formulations are either solids, usually known as wettable powers, or liquids, usually known as emulsifiable concentrates, or aqueous suspensions. The present invention contemplates all vehicles by which the compounds of this invention can be formulated for delivery with a fungicide. As will be readily appreciated, any material to which these compounds can be added may be used, provided they yield the desired utility without significant interference with activity of the compounds of this invention.

Wettable powders, which may be compacted to form water dispersible granules, comprise an intimate mixture of the active compound, an inert carrier, and surfactants. The concentration of the active compound is usually from about 1% to about 95% w/w/, more preferably about 1% to about 50% w/w. In the preparation of wettable powder compositions, the active compound can be compounded with any of the finely divided solids, such as prophyllite, talc, chalk, gypsum, Fuller's earth, bentonite, attapulgite, starch, casein, gluten, montmorillonite clays, diatomaceous earths, purified silicantes or the like. In such operations, the finely divided carrier is ground or mixed with the active compound in a volatile organic colvent. Effective surfactants, comprising from about 0.5% to about 10% of the wettable powder, include sulfonated lignins, naphthalenesulfonates, alkylbenzenesulfonates, alkyl sulfates, and non-ionic surfactants such as ethylene oxide adducts of alkyl phenols.

Emulsifiable concentrates of the compounds of this invention comprise a convenient concentration, such as from about 10% to about 50% w/w, in a suitable liquid. The compounds are dissolved in an inert carrier, which is either a water-miscible solvent or a mixture of water-immiscible organic solvents and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used such as, for example, terpenic solvents including rosin derivatives, aliphatic ketones, such as cyclohexanone, and complex alcohols such as 1-ethoxyethanol.

Emulsifiers which can be advantageously employed herein can be readily determined by those skilled in the art and include various non-ionic, anionic, cationic, and amphoteric emulsifiers, or a blend of two or more emulsifiers. Examples of non-ionic emulsifiers useful in preparing the emulsifiable concentrates include the polyalkylene glycol ethers and condensation products of alkyl and aryl phenols, aliphatic alcohols, aliphatic amines, or fatty acids with ethylene oxide, propylene oxides such as the ethoxylated alkyl phenols, and carboxylic esters solubilised with polyol or polyoxyalkylene. Cationic emulsifiers include quaternary ammonium compounds and fatty amine salts. Anioic emulsifiers include the oil-soluble salts (e.g., calcium) of alkylaryl sulfonic acids, oil-soluble sals of sulphated polyglycol ethers, and appropriate salts of phosphated polyglycol ether.

Representative organic liquids which can be employed in preparing the emulsifiable concentrates of the present invention are the aromatic liquids such as xylene, propylbenzene fractions or mixed naphthalene fractions, mineral oils, substituted aromatic organic liquids such as dioctyl phthalate, kerosene, and dialkyl amides of various fatty acids; particularly the dimethyl amides of fatty glycols and glycol derivatives such as the n-butyl ether, ethyl ether, or methyl ether of triethylene glycol. Mixtures of two or more organic liquids are also often suitably employed in the preparation of the emulsifiable concentrate. The preferred organic liquids are xylene and propylbenzene fractions, with xylene being most preferred. The surface active dispersing agents are usually employed in liquid compositions and in the amount of from 0.1 to 20 percent by weight of the combined weight of the dispersing agent and active compound. The active compositions can also contain other compatible additives, for example, plant growth regulators and other biologically active compounds used in agriculture. It is envisioned that substances that induce encystment of zoospores, such as pectin, a metal ion, and an inorganic compound or inorganic salt compound selected from the group consisting of Ca, Zn, Mg, Mn, NaNO₃, KNO₃, and NaCl, may be added to compositions containing a fungicide and a zoospore attractant derivative to further improve disease control.

Aqueous suspensions comprise suspensions of water-insoluble compounds of this invention, dispersed in an aqueous vehicle at a concentration in the range from about 5% to about 50% w/w. Suspensions are prepared by finely grinding the compound and vigorously mixing it into a vehicle comprised of water and surfactants chosen from the same types of above-discussed. Inert ingredients, such as inorganic salts and synthetic or natural gums, may also be added to increase the density and viscosity of the aqueous vehicle. It is often most effective to grind and mix the compound at the same time by preparing the aqueous mixture and homogenizing it in an implement such as a sand mill, ball mill, or piston-type homogenizer.

The compounds may also be applied as granular compositions which are particularly useful for applications to the soil. Granular compositions usually contain from about 0.5% to about 10% w/w of the compound dispersed in an inert carrier which consists entirely or in large part of coarsely divided attapulgite, bentonite, diatomite, clay, or a similar inexpensive substance. Such compositions are usually prepared by dissolving the compound in a suitable solvent and applying it to a granular carrier which has been preformed to the appropriate particle size, in the range of from about 0.5 to about 3 mm. Such compositions may also be formulated by making a dough or paste of the carrier and compound, and crushing, and drying to obtain the desired granular particle.

Dusts containing the compounds are prepared simply by intimately mixing the compound in powdered form with a suitable dusty agricultural carrier such as, for example, kaolin clay, ground volcanic rock, and the like. Dusts can suitably contain from about 1% to about 10% w/w of the compound.

The active compositions may contain adjuvant surfactants to enhance deposition, wetting, and penetration of the compositions onto the target crop and organism. These adjuvant surfactants may optionally be employed as a component of the formulation or as a tank mix. The amount of adjuvant surfactant will vary from 0.01 percent to 1.0 percent v/v based on a spray-volume of water, preferably 0.05 to 0.5 percent. Suitable adjuvant surfactants include ethoxylated nonyl phenols, ethoxylated synthetic or natural alcohols, salts of the esters of sulphosuccinic acids, ethoxylated organosilicones, ethoxylated fatty amines, and blends of surfactants with mineral or vegetable oils.

The composition may optionally include fungicidal combinations which comprise at least 1% of one or more of the compounds of this invention with another pesticidal compound. Such additional pesticidal compounds may be fungicides, insecticides, nematocides, miticides, arthropodicides, bactericides or combinations thereof that are compatible with the compounds of the present invention in the medium selected for application, and not antagonistic to the activity of the present compounds. Accordingly, in such embodiments, the other pesticidal compound is employed as a supplemental toxicant for the same or for a different pesticidal use. The compounds in combination can generally be present in a ratio of from 1:10 to 100:1.

The present invention includes within its scope methods for the control or prevention of fungal attack. These methods comprise applying to the locus of the fungus, or to a locus in which the infestation is to be prevented (for example, applying to potato, tomato, cucurbit or grape plants), an effective amount of one or more of the compounds of this invention and an agriculturally effective amount of a fungicide active on oomycete fungi. The compounds of this invention are suitable for treatment of various plants while exhibiting low phytotoxicity. The compounds of this invention are applied by any of a variety of known techniques, either as the compounds or as compositions including the compounds. For example, the compounds may be applied to plant foliage, blossoms, stems, fruits, the area adjacent to the plant, soil, seeds, germinating seeds, roots, liquid and solid growth media, and hydroponic growth solutions for the control of various fungi without damaging the commercial value of the plants. The materials are applied in the form of any of the generally used formulation types, for example, as solutions, dusts, wettable powders, flowable concentrates, or emulsifiable concentrates. These materials are conveniently applied in various known fashions.

The compounds of this invention have broad ranges of efficacy in fungicide formulations. The exact amount of the zoospore attractant derivative to be applied is dependent not only on the specific zoospore attractant derivative being applied, but also on the particular action desired, the fungal species to be controlled and the stage of growth thereof, as well as the part of the plant or other product to be contacted with the fungicidally effective ingredient. Thus, the compounds of this invention, and compositions containing the same, may not be equally effective at similar concentrations or against the same fungal species. The compounds of this invention and compositions thereof in mixtures with fungicides are effective on plants in a disease inhibiting and phytologically acceptable amounts.

While the novel technology has been illustrated and described in detail in the figures and foregoing description, the same is to be considered as illustrative and not restrictive in character, it being understood that only the variously exemplary embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

1. A composition selected from the group consisting of Formula 1, Formula 2,
Formula 3, Formula 4, Formula 5, and Formula 6 wherein,
Formula 1 is 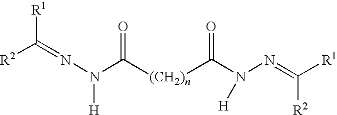
wherein,
R₁=iso-butyl, sec-butyl, or tert-butyl-CH₂;
R₂═H; and
n=0-25;
Formula 2 is 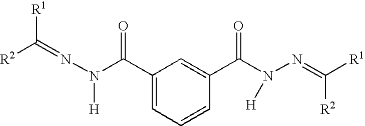
wherein,
R₁=sec-butyl, or tert-butyl-CH₂; and
R₂═H;
Formula 3 is 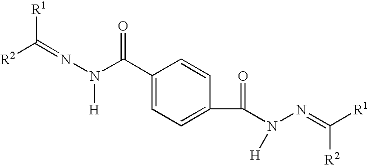
wherein,
R₁=iso-butyl, sec-butyl, or tert-butyl-CH₂; and
R₂═H;
Formula 4 is 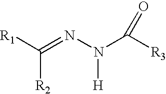
wherein,
R₁=iso-butyl-,
R₂═H; and
R₃=an n-alkyl including 2-25 carbons, excluding n-heptyl or n-undecyl alkyls, or a branched-alkyl including 4-25 carbons, or a substituted or unsubstituted cycloalkyl including 3-25 carbons, or a substituted or unsubstituted arylalkyl including 12-26 carbons; or
R₁=sec-butyl, or tert-butyl-CH₂;
R₂═H; and
R₃=an n-alkyl including 1-25 carbons, or a branched-alkyl including 3-25 carbons, or a substituted or unsubstituted cycloalkyl including 7-25 carbons, or a substituted or unsubstituted arylalkyl including 7-25 carbons, or
R₁=iso-butyl,
R₂=methyl; and
R₃=an n-alkyl including 2, 3, 6 and 12-25 carbons, or a branched-alkyl including 3-25 carbons, or a substituted or unsubstituted cycloalkyl including 3-25 carbons, or a substituted or unsubstituted arylalkyl including 7-25 carbons;
Formula 5 is 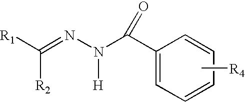
wherein,
R₄═H, alkyl or haloalkyl or alkoxy or alkylthio, or haloalkoxy or haloalkylthio, each including 1-4 carbons, or halo, hydroxyl, nitro, carboxyl acid, carboxylic acid derivatives or cyano; and either
R₁=sec-butyl, or tert-butyl-CH₂; and
R₂═H; or
R₁=iso-butyl; and
R₂=methyl; and
Formula 6 is 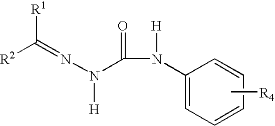
wherein;
R₄═H, alkyl or haloalkyl or alkoxy or alkylthio, or haloalkoxy or haloalkylthio, each including 1-4 carbons, or halo, hydroxyl, nitro, carboxyl acid, carboxylic acid derivatives or cyano; and either
R₁=iso-butyl, sec-butyl, or tert-butyl-CH₂; and
R₂═H; or
R₁=iso-butyl; and
R₂=methyl.

2. The composition of claim 1, wherein R1 is iso-butyl and R2 is hydrogen in Formulae 1, 3, 4, and 6.

3. A method whereby the compositions of claim 1 are used to attractant zoospores of oomycete fungi.

4. The method of claim 3 in which the zoospores are spores of at least one fungi selected from the group consisting of Phytophthora infestans, Plasmopara viticola, Phytophthora capsici, Pseudoperonospora cubensis, Bremia lactucae, Phytophthora phaseoli, Phytophthora nicotiane var. parasitica, Sclerospora graminicola, Sclerophthora rayssiae, Phytophthora palmivora, Phytophthora citrophora, Sclerophthora macrospora, Sclerophthora graminicola, Phytophthora cactorum, Phytophthora syringe, Pseudoperonospora humuli, and Albugo candida.

5. The method of claim 3 in which the zoospores are spores of at least one fungi selected from the group consisting of Phytophthora infestans, Plasmopara viticola, Phytophthora capsici, and Pseudoperonospora cubensis.

6. A method for controlling a fungal pathogen, comprising the steps of:
providing at least one composition according to claim 1, and
applying an agriculturally effective amount of one or more fungicides applied to plants.

7. A formulation for the control of a fungus, comprising:
at least one composition according to claim 1, and
at least one fungicide.

8. The formulation according to claim 7, wherein the fungicide is effective against a fungus that produces motile zoospores.

9. The formulation according to claim 8, wherein the fungicide is selected from the group consisting of mancozeb, maneb, zineb, thiram, propineb, metiram, copper hydroxide, copper oxychloride, Bordeaux mixture, captan, folpet, amisulbrom, azoxystrobin, trifloxystrobin, picoxystrobin, kresoxim-methyl, fluoxastrobin, pyraclostrobin, famoxadone, fenamidone, metalaxyl, mefenoxam, benalaxyl, cymoxanil, propamocarb, dimethomorph, flumorph, mandipropamid, iprovalicarb, benthiavalicarb-isopropyl, valiphenal, zoxamide, ethaboxam, cyazofamid, fluopicolide, fluazinam, chlorothalonil, dithianon, tolylfluanid, 4-fluorophenyl(1S)-1-({[(1R,S)-(4-cyanophenyl)ethyl]sulfonyl}methyl)propylcarbamate and triazolopyrimidine compounds of Formula I: 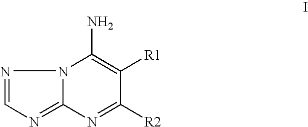
wherein R1 is ethyl, 1-octyl, 1-nonyl, or 3,5,5-trimethyl-1-hexyl and R2 is methyl, ethyl, 1-propyl, 1-octyl, trifluoromethyl, or methoxymethyl.

10. A method for controlling a fungal infestation, comprising the steps of:
providing at least one formulation according to claim 9; and
applying an agriculturally effective amount of the formulation to susceptible plants or an area adjacent to a fungus.

11. The formulation according to claim 7, wherein the composition further includes at least one zoospore attractant derivative.

12. The composition of claim 7, wherein the fungicide is a non-copper based fungicide.

13. The composition of claim 7, wherein the fungicide is selected to control diseases caused by oomycete fungal pathogens selected from the group consisting of Phytophthora infestans, Plasmopara viticola, Phytophthora capsici, Pseudoperonospora cubensis Bremia lactucae, Phytophthora phaseoli, Phytophthora nicotiane var. parasitica, Sclerospora graminicola, Sclerophthora rayssiae, Phytophthora palmivora, Phytophthora citrophora, Sclerophthora macrospora, Sclerophthora graminicola, Phytophthora cactorum, Phytophthora syringe, Pseudoperonospora humuli, and Albugo candida.

14. A method of controlling plant diseases caused by oomycete fungal pathogens including the steps of:
providing a formulation including the composition of claim 7, and
applying an agriculturally effective amount of the formulation to at least one of the following: the plant, plant foliage, blossoms, stems, fruits, the area adjacent to the plant, soil, seeds, germinating seeds, roots, liquid and solid growth media, and hydroponic growth solutions.

15. The method of claim 14 wherein the plant is a grape, potato, tomato, cucumber, squash or other cucurbits, cabbage or other crucifer, lettuce, beans, corn, soybeans, pepper or hops.

16. The formulation according to claim 11, wherein the zoospore attractant derivative releases a C4-C8 aldehyde selected from the group consisting of isovaleraldehyde, 2-methylbutyraldehyde, valeraldehyde, isobutyraldehyde, butyraldehyde, 4-methylpentanal, 3,3-dimethylbutyraldehyde, 3-methylthiobutyraldehyde, 2-cyclopropylacetaldehyde, 3-methylcrotonaldehyde, 2-ethylcrotonaldehyde, crotonaldehyde, 2-methylcrotonaldehyde, 3-indolecarbaldehyde, furfural (2-furaldehyde), 2-thiophenecarboxaldehyde, 2-ethylbutyraldehyde, cyclopropanecarboxaldehyde, 2,3-dimethylvaleraldehyde, 2-methylvaleraldehyde, tetrahydrofuran-3-carboxaldehyde, and cyclopentanecarboxaldehyde.

17. The formulation according to claim 11, wherein the zoospore attractant derivative releases a C4-C8 ketone.
